(19) United States
(12) Patent Application Publication
(10) Pub. No.: US 2010/0266954 A1
(43) Pub. Date: Oct. 21, 2010

(54) ADAMANTANE DERIVATIVE, METHOD FOR PRODUCING THE SAME, AND CURING COMPOSITION CONTAINING ADAMANTANE DERIVATIVE

(75) Inventors: Katsuki Ito, Chiba (JP); Shinji Tanaka, Chiba (JP); Naoya Kawano, Chiba (JP); Hideki Yamane, Chiba (JP); Hidetoshi Ono, Chiba (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Idemitsu Kosan Co., Ltd., Tokyo (JP)

(21) Appl. No.: 12/742,379

(22) PCT Filed: Nov. 10, 2008
(86) PCT No.: PCT/JP2008/070419

§ 371 Date: May 11, 2010

(30) Foreign Application Priority Data
Nov. 13, 2007 (JP) 2007 294728

(51) Int. Cl. G03F 7/004; G03F 7/20; C07C 69/608; C08F 20/10

(52) U.S. Cl. 430/285.1; 430/325; 560/220; 526/282; 560/193

(57) ABSTRACT

An adamantane derivative capable of affording a cured product which is excellent in optical characteristics such as transparency and light resistance, durability such as long-term heat resistance, and electrical characteristics such as dielectric constant, a process for producing such an adamantane derivative, and a curable composition containing such an adamantane derivative, the adamantane derivative being represented by the general formula (I) shown below and having a group selected from an acrylate group, a methacrylate group and a trifluoromethacrylate group,

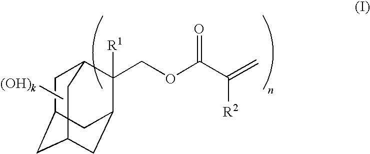

where R¹ represents a group selected from a hydroxyl group, an acrylate group, a methacrylate group and a trifluoromethacrylate group, R² represents a group selected from a hydrogen atom, a methyl group and a trifluoromethyl group, k is an integer of 0 to 4 and n is an integer of 1 to 6.

### TECHNICAL FIELD

The present invention relates to a novel adamantane derivative, to a method for producing the same and to a curing composition containing such an adamantane derivative. More specifically, the present invention is directed to an adamantane derivative capable of affording a cured product which shows excellent transparency, optical characteristics, durability and electrical characteristics and which is useful as a photoresist material for semiconductors, a color resist material, a sealant for opto-semiconductors, an optical electronic member, an adhesive agent for them, etc., to a method for producing such an adamantane derivative, and to a curing composition containing such an adamantane derivative.

### BACKGROUND ART

Adamantane is a stable, highly symmetrical compound in which four cyclohexane rings are condensed to form a cage-like structure. It is known that adamantane derivatives, which show peculiar functions, are useful as raw materials for medical materials and highly functional industrial materials. Further, because adamantane has specific optical characteristics and heat resistance, an attempt has been made to use it as, for example, an optical disc substrate, an optical fiber or a lens (see, for example, Patent Documents 1 and 2). Another attempt has also been made to use an adamantane ester as a raw material for a photoresist resin by utilizing its sensitivity to an acid, dry etching resistance and transparency to UV rays (see, for example, Patent Document 3).

An acrylic resin having excellent transparency and light resistance has been hitherto generally used as a resin for use in optical members. In recent years, however, as high intensity laser light, blue light and near ultra violet light are increasingly used in the field of optoelectronic devices, there is a demand for a resin having more excellent transparency, heat resistance and light resistance than hitherto.

On the other hand, studies have been also made for overcoming the drawback (for improving heat resistance) of acrylic resins which are excellent in optical characteristics. Thus, studies have been made on cross-linked acrylic resins using polyfunctional acrylate monomers. In particular, a number of techniques concerning acrylate copolymers containing alicyclic acrylate units have been proposed in view of the fact that a cured product of alicyclic acrylate has a high glass transition temperature, low curing shrinkage and low moisture absorbing properties. For example, there is disclosed a resin composition which includes a (meth)acrylate, as a monomer component A, having a C₄ or lower aliphatic hydrocarbon group in its ester moiety, an alicyclic polyfunctional (meth)acrylate, as a monomer component B, and a polymerization initiator and which is curable by heat or light (for example, Patent Document 4). Also proposed is a composition for use in optical adhesive agent or the like application which includes a (meth)acrylic acid ester having a C₅ to C₂₂ alicyclic hydrocarbon group in its ester moiety, and a polyfunctional (meth)acrylate having an alkylene oxide (for example, Patent Document 5). Although the heat resistance of the composition required as an adhesive at the time of mounting is satisfactory, the composition is still insufficient with respect to the heat resistance and mechanical properties required as a structural component.

Accordingly, in acrylic-based curable compositions, it is desired to provide a composition which can give a cured product having high optical transparency, excellent light resistance, excellent heat resistance, excellent mechanical properties and low curing shrinkage and suited as optical parts.

Patent Document 1: Japanese Patent Application Publication No. Hei 06-305044

Patent Document 2: Japanese Patent Application Publication No. Hei 09-302077

Patent Document 3: Japanese Patent Application Publication No. Hei 04-39665

Patent Document 4: Japanese Patent Application Publication No. 2006-193660

Patent Document 5: Japanese Patent Application Publication No. Hei 11-61081

### DISCLOSURE OF THE INVENTION

### Problem to be Solved by the Invention

The objective problem of the present invention is to provide an adamantane derivative capable of affording a cured product which shows excellent transparency, optical characteristics, durability and electrical characteristics and which is useful as a photoresist material for semiconductors, a color resist material, a sealant for opto-semiconductors, an optical electronic member and an adhesive agent for them; a process for producing such an adamantane derivative; and a curable composition, a (meth)acrylic-based polymer and a resist composition each containing such an adamantane derivative.

### Means for Solving the Problem

The present inventors have made an earnest study and, as a result, have found that the above problem can be solved by using an adamantane derivative having a specific structure. The present invention has been completed based on such a finding.

That is, the present invention provides as follows:

1. An adamantane derivative represented by the following general formula (I):

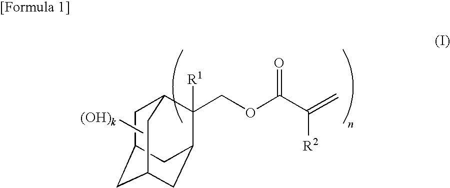

[wherein R¹ represents a group selected from a hydroxyl group, an acrylate group, a methacrylate group and a trifluoromethacrylate group, R² represents a group selected from a hydrogen atom, a methyl group and a trifluoromethyl group, k is an integer of 0 to 4 and n is an integer of 1 to 6];
2. The adamantane derivative as recited in above 1, wherein k in the general formula (I) is zero;
3. A process for producing the adamantane derivative according to above 1, comprising reacting an epoxyadamantane with a compound selected from acrylic acid, methacrylic acid, trifluoromethacrylic acid, acrylic anhydride, methacrylic anhydride and trifluoromethacrylic anhydride;
4. A curable composition comprising the adamantane derivative according to above 1, and a polymerization initiator;
5. A cured product obtained by curing the curable composition according to above 4 by heating or by light irradiation;
6. A photoresist material using the adamantane derivative according to above 1;
7. A color resist material using the adamantane derivative according to above 1;
8. A (meth)acrylic-based polymer containing a monomer unit based on the adamantane derivative according to above 1;
9. A resist composition comprising the (meth)acrylic polymer according to above 8; and
10. A resist pattern forming method, comprising the steps of forming a resist film on a substrate using the resist composition according to claim 9, selectively light-exposing the resist film, and subjecting the selectively light-exposed resist film to an alkali development treatment to form a resist pattern.

### EFFECT OF THE INVENTION

The curable composition containing an adamantane derivative of the present invention having a group selected from an acrylate group, a methacrylate group and a trifluoromethacrylate group is capable of affording a cured product which is excellent in optical characteristics such as transparency and light resistance, durability such as long-term heat resistance and etching resistance, and electrical characteristics such as dielectric constant, and which is usable as a color resist material, as a sealant for opto-semiconductors, as an optical electronic member (such as an optical waveguide, a lens for optical communication and an optical film), as an adhesive agent for them, and also as a material for forming semiconductors such as a photoresist material for semiconductors and a anti-reflection coating for semiconductors.

The use of a (meth)acrylic-based polymer containing, as its monomer units, an adamantane derivative of the present invention can provide a resist composition which is excellent in acid diffusion property, solubility in a developer liquid and the like properties.

### BRIEF DESCRIPTION OF DRAWINGS

FIG. 1 is a graph showing normalized film thickness (−) as a function of exposure dose (mJ/cm²) according to an Example 6 of this invention.

### BEST MODE FOR CARRYING OUT THE INVENTION

### Adamantane Derivative and Process for its Production

An adamantane derivative of the present invention is an adamantane derivative represented by the general formula (I) shown below (hereinafter occasionally simply referred to as “adamantane derivative”) having a group selected from an acrylate group, a methacrylate group and a trifluoromethacrylate group:

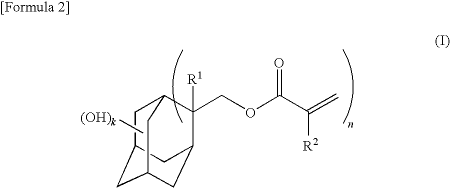

[where R¹ represents a group selected from a hydroxyl group, an acrylate group, a methacrylate group and a trifluoromethacrylate group, R² represents a group selected from a hydrogen atom, a methyl group and a trifluoromethyl group, k is an integer of 0 to 4 and n is an integer of 1 to 6].

As the adamantane derivative, a compound of the general formula (I) in which k is zero is particularly preferred.

The adamantane derivative represented by the above general formula (I) may be obtained by reacting an epoxyadamantane with a compound selected from acrylic acid, methacrylic acid, trifluoromethacrylic acid, acrylic anhydride, methacrylic anhydride and trifluoromethacrylic anhydride in the presence of a catalyst.

A reaction of an epoxyadamantane with a compound selected from acrylic acid, methacrylic acid and trifluoromethacrylic acid may be represented by the following reaction formula (a):

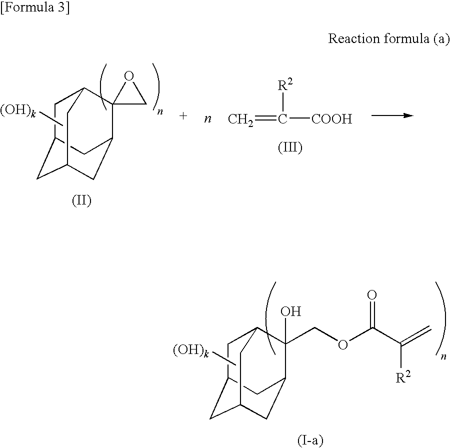

[where R², k and n are as defined above].

Thus, an adamantane derivative represented by the general formula (I-a) may be obtained by reacting an adamantanespirooxirane compound (II) with acrylic acid, methacrylic acid or trifluoromethacrylic acid (III).

On the other hand, a reaction of an epoxyadamantane with a compound selected from acrylic anhydride, methacrylic anhydride and trifluoromethacrylic anhydride may be represented by the following reaction formula (b):

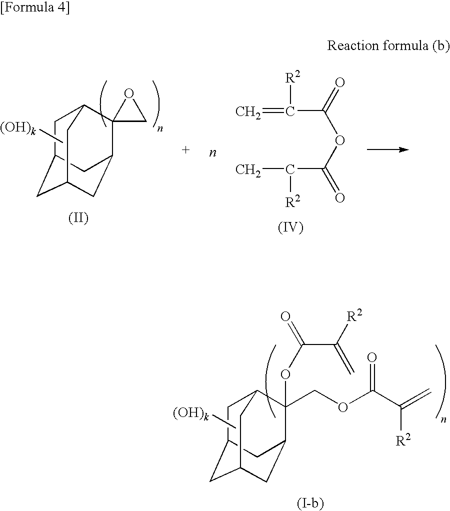

[where R², k and n are as defined above].

Thus, an adamantane derivative represented by the general formula (I-b) may be obtained by reacting an adamantanespirooxirane compound (II) with acrylic anhydride, methacrylic anhydride or trifluoromethacrylic anhydride (IV).

As the epoxyadamantane used as the starting material, there may be mentioned, for example, adamantane-2-spirooxirane, adamantane-2,4-di(spirooxirane), adamantane-2,4,6-tri(spirooxirane), adamantane-2,4,6,8-tetra(spirooxirane), adamantane-2,4,6,8,10-penta(spirooxirane), adamantane-2,4,6,8,10,12-hexa(spirooxirane), adamantane-1-hydroxy-2-spirooxirane, adamantane-1-hydroxy-2,4-di(spirooxirane), adamantane-1-hydroxy-2,4,6-tri(spirooxirane), adamantane-1-hydroxy-2,4,6,8-tetra(spirooxirane), adamantane-1-hydroxy-2,4,6,8,10-penta(spirooxirane), adamantane-1-hydroxy-2,4,6,8,10,12-hexa(spirooxirane), adamantane-1,3-dihydroxy-2-spirooxirane, adamantane-1,3-dihydroxy-2,4-di(spirooxirane), adamantane-1,3-dihydroxy-2,4,6-tri(spirooxirane), adamantane-1,3-dihydroxy-2,4,6,8-tetra(spirooxirane), adamantane-1,3-dihydroxy-2,4,6,8,10-penta(spirooxirane), adamantane-1,3-dihydroxy-2,4,6,8,10,12-hexa(spirooxirane), adamantane-1,3,5-trihydroxy-2-spirooxirane, adamantane-1,3,5-trihydroxy-2,4-di(spirooxirane), adamantane-1,3,5-trihydroxy-2,4,6-tri(spirooxirane), adamantane-1,3,5-trihydroxy-2,4,6,8-tetra(spirooxirane), adamantane-1,3,5-trihydroxy-2,4,6,8,10-penta(spirooxirane), adamantane-1,3,5-trihydroxy-2,4,6,8,10,12-hexa(spirooxirane), adamantane-1,3,5,7-tetrahydroxy-2-spirooxirane, adamantane-1,3,5,7-tetrahydroxy-2,4-di(spirooxirane), adamantane-1,3,5,7-tetrahydroxy-2,4,6-tri(spirooxirane), adamantane-1,3,5,7-tetrahydroxy-2,4,6,8-tetra(spirooxirane), adamantane-1,3,5,7-tetrahydroxy-2,4,6,8,10-penta(spirooxirane), adamantane-1,3,5,7-tetrahydroxy-2,4,6,8,10,12-hexa(spirooxirane).

Illustrative of preferred epoxyadamantanes are adamantane-2-spirooxirane, adamantane-2,4-di(spirooxirane), adamantane-1-hydroxy-2-spirooxirane, adamantane-1-hydroxy-2,4-di(spirooxirane), adamantane-1,3-dihydroxy-2-spirooxirane, adamantane-1,3-dihydroxy-2,4-di(spirooxirane), adamantane-1,3,5-trihydroxy-2-spirooxirane, adamantane-1,3,5-trihydroxy-2,4-di(spirooxirane), adamantane-1,3,5,7-tetrahydroxy-2-spirooxirane and adamantane-1,3,5,7-tetrahydroxy-2,4-di(spirooxirane).

Above all, adamantane-2-spirooxirane and adamantane-2,4-di(spirooxirane) are particularly preferred.

In the case of the reaction of the above reaction formula (a), the proportion of the adamantanespirooxirane compound (II) and the acrylic acid, methacrylic acid or trifluoromethacrylic acid (III) is preferably such that the amount of the acrylic acid, methacrylic acid or trifluoromethacrylic acid is 1 to 5 moles, more preferably 1 to 3 moles, per mole of the spirooxirane group of the compound (II) from the viewpoint of the post treatment.

In the case of the reaction of the above reaction formula (b), the proportion of the adamantanespirooxirane compound (II) and the acrylic anhydride, methacrylic anhydride or trifluoromethacrylic anhydride (IV) is preferable such that the amount of the acrylic anhydride, methacrylic anhydride or trifluoromethacrylic anhydride is 2 to 10 moles, more preferably 2 to 4 moles, per mole of the spirooxirane group of the compound (II) from the viewpoint of the post treatment.

The reaction of an epoxyadamantane with a compound selected from acrylic acid, methacrylic acid, trifluoromethacrylic acid, acrylic anhydride, methacrylic anhydride and trifluoromethacrylic anhydride is generally carried out in the presence of a catalyst. As the catalyst, there may be mentioned, for example, sodium amide, triethylamine, tributylamine, trioctylamine, pyridine, lutidine, dimethylaminopyridine, N,N-dimethylaniline, 1,5-diazabicyclononene-5 (DBN), 1,8-diazabicycloundecene-7 (DBU), tetramethylammonium chloride, tetraethylammonium chloride, sodium, potassium, cesium, sodium hydride, potassium hydride, sodium hydroxide, potassium hydroxide, sodium phosphate, potassium phosphate, sodium carbonate, potassium carbonate, cesium carbonate, silver oxide, sodium methoxide and potassium t-butoxide. Illustrative of preferred catalysts are dimethylaminopyridine, DBN, DBU and tetraethylammonium bromide.

The using amount of the catalyst is generally about 0.01 to about 2 moles, preferably 0.01 to 1 mole, relative to the epoxyadamantane used as the starting material. When the using amount of the catalyst is 0.01 mole or more, the reaction time becomes not too long and is moderate. When the using amount of the catalyst is 2 moles or less, a good balance between the obtained effect and economy may be achieved.

The reaction may be carried out without using a solvent. If necessary, however, a solvent may be used. A solvent capable of dissolving the epoxyadamantane in an amount of preferably 0.5 wt % or more, more preferably 10 wt % or more, is suitably used. Specific examples of the solvent include hexane, heptane, toluene, dimethylformamide (DMF), dimethylacetamide (DMAc), dimethyl sulfoxide (DMSO), ethyl acetate, diethyl ether, tetrahydrofuran, acetone, methyl ethyl ketone, methyl isobutyl ketone. These solvents may be used singly or in combination. Among these solvents, DMF and DMSO are preferred.

The solvent is preferably used in such an amount that the concentration of the epoxyadamantane therein is 0.5 wt % or more, more preferably 10 wt % or more. In this case, the epoxyadamantane in the solvent may be in a suspended state, but is desirably in a dissolved state.

The reaction of an epoxyadamantane with a compound selected from acrylic acid, methacrylic acid, trifluoromethacrylic acid, acrylic anhydride, methacrylic anhydride and trifluoromethacrylic anhydride is generally carried out at a temperature of about 0 to about 200° C., preferably 20 to 150° C. Too low a temperature causes a reduction of the reaction rate so that the reaction time becomes long. When the temperature is 0° C. or higher, the reaction rate is not reduced and is moderate so that the reaction time may be shortened. When the temperature is higher than 200° C., significant coloring of the product may occur.

The reaction pressure is about 0.01 to about 10 MPa, preferably from ambient pressure to 1 MPa, in terms of absolute pressure. Too high a reaction pressure causes a safety problem and is industrially disadvantageous because a special equipment must be used. A reaction pressure of 10 MPa or less is industrially advantageous because the safety is secured so that no special equipment is needed. The reaction time is generally about 1 minute to about 24 hours, preferably 1 to 15 hours.

The reaction product may be purified, if necessary, by, for example, distillation, crystallization or column separation. The purification method may be suitably selected in accordance with properties of the reaction product and kind of impurities.

As the preferred examples of the thus obtained adamantane derivative represented by the general formula (I), there may be mentioned (2-hydroxy-2-adamantyl)methyl-2-methacrylate and [2-(methacryloxy)-2-adamantyl]methyl methacrylate.

### Curable Composition:

The curable composition of the present invention contains the adamantane derivative represented by the above general formula (I) and a polymerization initiator. As the polymerization initiator, a thermal polymerization initiator is used when the composition is cured by heating, and a photopolymerization initiator is used when the composition is cured by light.

As the thermal polymerization initiator, there may be mentioned, for example, organic peroxides such as benzoyl peroxide, methyl ethyl ketone peroxide, methyl isobutyl peroxide, cumene hydroperoxide, t-butyl hydroperoxide and azo type initiators such as azobisisobutylonitrile.

As the photopolymerization initiator, there may be mentioned, for example, acetophenones, benzophenones, benzyls, benzoin ethers, benzyl diketals, thioxanthones, acylphosphine oxides, acylphosphinate esters, aromatic diazonium salts, aromatic sulfonium salts, aromatic iodonium salts, aromatic iodosyl salts, aromatic sulfoxonium salts and metallocene compounds.

The using amount of the polymerization initiator is generally preferably 0.01 to 4 parts, more preferably 0.5 to 2 parts, per 100 parts of the above-described adamantane derivative. When the content of the polymerization initiator is in the above range, suitable polymerization and suitable physical properties such as optical properties may be achieved.

The curable composition containing the adamantane derivative of the present invention and the polymerization initiator may be compounded, if necessary, with various kinds of conventionally employed known additives such as a curing accelerator, a decay-preventing agent, a modifying agent, a silane coupling agent, a defoaming agent, an inorganic powder, a solvent, a leveling agent, a mold-release agent, a dye and a pigment.

The curing accelerator is not particularly limited. Examples of the curing accelerator include 1,8-diazabicycloundecene-7; tertiary amines such as triethylenediamine and tris(2,4,6-dimethylaminomethyl)phenol; imidazoles such as 2-ethyl-4-methylimidazole and 2-methylimidazole; phosphorus compounds such as triphenylphosphine, tetraphenylphosphonium bromide, tetraphenylphosphonium tetraphenylborate, tetra-n-butylphosphonium O,O-diethylphosphorodithioate; quaternary ammonium salts; organometallic salts; and derivatives thereof. These compounds may be used singly or in combination. Among the above curing accelerators, the tertiary amines, imidazoles and phosphorus compounds are preferred.

The content of the curing accelerator is preferably 0.01 to 8.0 parts, more preferably 0.1 to 3.0 parts, per 100 parts of the curing composition containing the adamantane derivative and the polymerization initiator. When the content of the curing accelerator is in the above range, sufficient curing accelerating effect may be obtained with no coloring of the cured product being accompanied.

As the decay-preventing agent, there may be mentioned those conventionally known ones such as a phenolic compound, an amine compound, an organic sulfur compound and a phosphorus compound. Addition of such a decay-preventing agent may enable maintenance of properties such as heat resistance and transparency of the curable composition of the present invention.

Examples of the phenolic compound include commercially available products such as Irganox 1010 (trademark, manufactured by Ciba Specialty Chemicals Inc.), Irganox 1076 (trademark, manufactured by Ciba Specialty Chemicals Inc.), Irganox 1330 (trademark, manufactured by Ciba Specialty Chemicals Inc.), Irganox 3114 (trademark, manufactured by Ciba Specialty Chemicals Inc.), Irganox 3125 (trademark, manufactured by Ciba Specialty Chemicals Inc.), Irganox 3790 (trademark, manufactured by Ciba Specialty Chemicals Inc.), BHT, Cyanox 1790 (trademark, Manufactured by Cyanamid Co.) and Sumilizer GA-80 (trademark, manufactured by Sumitomo Chemical Co., Ltd.).

Examples of the amine compound include Irgastab FS042 (trademark, manufactured by Ciba Specialty Chemicals Inc.), GENOX EP (trademark, manufactured by Crompton Corp., chemical name: dialkyl-N-methylamine oxide) and hindered amines such as ADK STAB LA-52, LA-57, LA-62, LA-63, LA-67, LA-68, LA-77, LA-82, LA-87, and LA-94, all manufactured by ADEKA Corporation, Tinuvin 123, 144, 440, 662, Chimassorb 2020, 119, and 944, all manufactured by Ciba Specialty Chemicals Inc., Hostavin N30 manufactured by Hoechst GmbH, Cyasorb UV-3346 and UV-3526, both manufactured by Cytec Industries Inc., Uval 299 manufactured by Great Lakes Chemical Corp., and Sanduvor PR-31 manufactured by Clariant Corp.

Examples of the organic sulfur compound include commercially available products such as DSTP (Yoshitomi) (trademark, manufactured by Yoshitomi Pharmaceutical Co., Ltd.), DLTP (Yoshitomi) (trademark, manufactured by Yoshitomi Pharmaceutical Co., Ltd.), DLTOIB (trademark, manufactured by Yoshitomi Pharmaceutical Co., Ltd.), DMTP (Yoshitomi) (trademark, manufactured by Yoshitomi Pharmaceutical Co., Ltd.), Seenox 4125 (trademark, manufactured by Shipro Kasei, Ltd.) and Cyanox 1212 (trademark, manufactured by Cyanamid Co.).

As the modifying agent there may be mentioned, for example, conventionally known ones such as glycols, silicones, and alcohols. Examples of the silane coupling agent include conventionally known ones such as silane-type and titanate-type silane coupling agents. As the defoaming agent there may be mentioned conventionally known ones such as a silicone type defoaming agent. As the inorganic powder there may be used those having a particle diameter of several nm to 10 μm depending on the intended use. Examples of the inorganic powder include known inorganic powder such as glass powder, silica powder, titania, zinc oxide and alumina. A solvent may be used when the curing component is in the form of powder. A solvent may be also used as diluent for coating. Examples of the solvent include aromatic solvents such as toluene and xylene and ketone solvents such as methyl ethyl ketone, methyl isobutyl ketone and cyclohexanone.

### Cured Product:

The cured product of the present invention is one which is obtained by curing the curable composition containing the adamantane derivative represented by the above general formula (I) and the polymerization initiator.

As a method for curing the curable composition, there may be used, for example, a curing method in which the curable composition, after having been added with optionally usable various additives, is charged into a mold (resin mold) or formed into a desired shape by coating, and, thereafter, cured by heating or light irradiation.

In the case of thermal curing by heating, the curing temperature is generally about 50 to about 200° C., preferably 100 to 180° C. When the temperature is 50° C. or higher, curing failure does not occur. When the temperature is 200° C. or lower, coloring does not occur. The curing time varies with the curing components, curing agent, curing accelerator and initiator used, but is preferably 0.5 to 6 hours.

In the case of curing by light irradiation, UV rays are used. The irradiation amount of UV rays is generally about 500 to about 5,000 mJ/cm², preferably 1,000 to 4,000 mJ/cm².

If desired, UV ray irradiation may be followed by heating. The heating is preferably performed at 70 to 200° C. for 0.5 to 12 hours.

The physical properties of the thus obtained cured product are such that the curing shrinkage is preferably 15% or less, heat resistance in terms of glass transition temperature is 100° C. or higher, the mechanical strength in terms of bending strength is 2.0 MPa or more at 250° C., and saturated moisture absorption rate (in mass) of 1.5% or less at 85° C./85%.

The cured product obtained by curing the curable composition of the present invention, which has excellent heat resistance, transparency, light resistance, etc., is suitably used as a sealant and an adhesive for an optical semiconductor (such as LED), a flat panel display (such as organic EL device), an electronic circuit, and an optical circuit (an optical waveguide); and as an optical electronic member such as a lens for optical communication and an optical film. A molding method for the production of them is not particularly restricted and may be injection molding, blow molding, press molding, etc. Preferably adopted is a production method in which a composition in the form of pellets is used for injection-molding with an injection molding equipment.

Thus, the cured product of the present invention having excellent properties may be used as a semiconductor element/an integrated circuit (such as an IC) and an individual semiconductor (such as a diode, a transistor and a thermistor) for use in an LED (such as an LED lamp, a chip LED, a light receiving element and a lens for an optical semiconductor), a sensor (such as a temperature sensor, a light sensor and a magnetic sensor), a passive component (such as a high frequency device, a resistor and a condenser), a structural component (such as a connector, a switch and a relay), an automobile part (such as a circuit system, a control system, a sensor and a lamp seal) and an adhesive (such as for an optical component, an optical disk and a pickup lens), and, in addition, as a surface coating for use in, for example, an optical film. Further, because the compound used for the cured product of the present invention is an adamantane derivative containing a group selected from an acrylate group, a methacrylate group and a trifluoromethacrylate group, the cured product is excellent in heat resistance and adhesiveness and additionally has good etching resistance. Therefore, the compound is useful for a sealant for photosemiconductors, a sealant for electronic circuits, an optical waveguide, a lens for optical communication, a sealant for organic EL devices, an optical film, a color resist, a sealant for semiconductors, a anti-reflection coating for semiconductors, and a photoresist material for use in the production of semiconductors. Thus the present invention also provides a photoresist material and a color resist material each using the adamantane derivative.

The constitution as a sealant for an optical semiconductor (LED, for example) may be applied to a device such as a bombshell type device or a surface mount type (SMT) device, may adhere well to semiconductors such as GaN formed on a metal or a polyamide, and further may be used by dispersing a fluorescent dye such as YAG in it. Furthermore, it may also be used for a surface coating material of a bombshell type LED and for a lens of a SMT type LED.

The constitution for an organic EL is applicable to an organic EL device having a structure of anode/hole-injection layer/luminescent layer/electron-injection layer/cathode which are formed in this order on a transparent substrate such as an ordinary glass or a transparent resin. As a sealant for an organic EL device, it may be used as an adhesive for covering an EL device with a metal can, a metal sheet, or a resin film coated with SiN, etc., or may be used for directly sealing an EL device after dispersing an inorganic filler in a curable composition containing the adamantane derivative of the present invention in order to impart a gas-barrier property to the curable composition. The constitution may be applied to a bottom emission type, which is currently a mainstream as a display system. However, the effects of transparency and heat resistance of the curable composition of the present invention may be advantageously utilized when the constitution is applied to a top emission type, which will draw attention in view of the light extraction efficiency in the future.

The constitution for an optical circuit is applicable to a thermo-optic switch of a single-mode and a multi-mode, an arrayed waveguide grating, an optical multiplexer or demultiplexer, a wavelength-variable filter, or a core material and a clad material for an optical fiber. It is also applicable to a micro lens array which focuses a light to a waveguide and to a mirror of an MEMS-type optical switch. Furthermore, it is also applicable to a dye binder for a photoelectric transducer.

The constitution for an optical film is applicable as a display of a film substrate for liquid crystal and a film substrate for organic EL, as a light diffusion film, as an anti-reflection film, and as a color-converting film in which a fluorescent dye is dispersed.

The constitution for a color resist is applicable as RGB constituting a color filter for a liquid crystal display and as a main ingredient or as an additive of a resist for forming a black matrix.

The present invention further provides a (meth)acrylic-based polymer containing a monomer unit based on the adamantane derivative of the present invention, and a resist composition containing the (meth)acrylic-based polymer.

### (Meth)acrylic-Based Polymer:

The (meth)acrylic-based polymer used for the resist composition of the present invention is preferably contains 5 to 90 mole %, more preferably 10 to 30 mole %, of monomer units based on the adamantane derivative represented by the above general formula (I).

The polymerization method for obtaining the (meth)acrylic polymer is not particularly limited and customarily employed polymerization methods may be adopted. Thus, conventionally known polymerization methods, for example, solution polymerization (boiling point polymerization, below boiling point polymerization), emulsion polymerization, suspension polymerization and mass polymerization can be adopted. It is preferred that the amount of the unreacted high boiling point monomer remaining in the reaction liquid after completion of the polymerization be as small as possible. Therefore, it is preferable to carry out an operation for removing the unreacted monomer, when necessary, during or after completion of the polymerization.

Among the above-described polymerization methods, a polymerization method using a radical polymerization initiator in a solvent is preferred. The polymerization initiator is not specifically limited, but a peroxide type polymerization initiator and an azo type polymerization initiator are suitably used.

As the peroxide type polymerization initiator, there may be mentioned organic peroxides such as a peroxycarbonate, a ketone peroxide, a peroxyketal, a hydroperoxide, a dialkyl peroxide, a diacyl peroxide, a peroxy ester (lauroyl peroxide and benzoyl peroxide). As the azo type polymerization initiator, there may be mentioned azo compounds such as 2,2′-azobis(isobutyronitrile), 2,2′-azobis(2-methylbutyronitrile), 2,2′-azobis(2,4-dimethylvaleronitrile) and dimethyl 2,2′-azobis(isobutyrate). The above polymerization initiators may be suitably used singly or in combination of two or more thereof according to the reaction conditions such as polymerization temperature.

Various methods may be adopted for removing the adamantane derivative of the present invention and other copolymerization monomers, which remain after the completion of the polymerization. From the viewpoint of processability and economy, a method for washing with a poor solvent against the acrylic polymer is preferred. Examples of the poor solvent against the acrylic polymer include methanol, ethanol, n-hexane, n-heptane and water.

The use of a mixed solvent composed of methanol and water can remove undesirable substances such as the unreacted monomer, polymerization initiator and reaction residues without or almost without removing molecules having a molecular weight of 300 or more. The mixing proportion of methanol and water is preferably such as to provide a mass ratio of methanol to water of 100:8 to 100:30, more preferably 100:10 to 100:20. The using amount of the washing solvent is preferably two times, preferably 4 to 8 times, the mass of the polymerization solvent from the viewpoint of removal efficiency of purities such as the unreacted monomer.

### Resist Composition:

The resist composition of the present invention is not specifically limited as long as it contains the above-described (meth)acrylic-based polymer. The content of the (meth)acrylic-based polymer of the present invention is preferably 2 to 50 parts, more preferably 5 to 15 parts, per 100 parts of the resist composition of the present invention.

The resist composition of the present invention may contain, in addition to the above-described (meth)acrylic-based polymer, a photoacid generator (PAG), a quencher such as an organic amine, an alkali-soluble component such as an alkali-soluble resin (for example, a novolak resin, a phenol resin, an imide resin and a carboxyl group-containing resin), a coloring agent (for example, a dye) and an organic solvent (for example, a hydrocarbon, a halogenated hydrocarbon, an alcohol, an ester, an ether, a cellosolve, a carbitol, a glycol ester and a mixed solvent thereof).

As the photoacid generator, customarily employed compounds capable of efficiently forming an acid by light exposure, such as a diazonium salt, an iodonium salt (for example, diphenyl iodonium hexafluorophosphate), a sulfonium salt (for example, triphenylsulfonium hexafluoroantimonate, triphenylsulfonium hexafluorophosphate, triphenylsulfonium nonafluorobutane sulfonate, and triphenylsulfonium methanesulfonate), a sulfonic acid ester [for example, 1-phenyl-1-(4-methylphenyl)sulfonyloxy-1-benzoylmethane, 1,2,3-trisulfonyloxymethylbenzene, 1,3-dinitro-2-(4-phenylsulfonyloxymethyl)benzene and 1-phenyl-1-(4-methylphenylsulfonyloxymethyl)-1-hydroxy-1-benzoylmethane], an oxathiazol derivative, s-triazine derivative, a disulfone derivative (for example, diphenyldisulfone), an imide compound, an oxime sulfonate, diazonaphthoquinone and benzoin tosylate, may be used. These photoacid generators may be used singly or in combination of two or more thereof.

The content of the photoacid generator in the resist composition of the present invention may be appropriately selected according to a strength of the acid generated by light exposure and a content of the monomer units based on the adamantane derivative in the (meth)acrylic-based polymer. For example, the photoacid generator is used in an amount of 0.1 to 30 parts by weight, preferably 1 to 25 parts by weight, still more preferably 2 to 20 parts by weight, per 100 parts by weight of the (meth)acrylic-based polymer.

The resist composition of the present invention may be prepared by mixing the (meth)acrylic-based polymer with a photoacid generator and, if necessary, an organic solvent, etc., the resulting mixture being optionally processed to remove impurities by customarily employed solid separation means such as a filter. The obtained resist composition is coated on a base or substrate and dried. The coating (resist film) is then exposed to light through a given mask (and further baked after the light exposure) to form a latent image pattern. This is then developed to form a fine pattern with a high degree of precision.

### Resist Pattern Forming Method:

The present invention further provides a resist pattern forming method, which includes the steps of forming a resist film on a substrate using the above-described resist composition, selectively light-exposing the resist film, and subjecting the selectively light-exposed resist film to an alkali development treatment to form a resist pattern.

As the substrate, there may be mentioned silicon wafers, metals, plastics, glasses and ceramics.

The step of forming a resist film using the resist composition may be carried out by using a conventional application means such as a spin coater, a dip coater and a roller coater. The applied film preferably has a thickness of, for example, 50 nm to 20 μm, more preferably 100 nm to 2 μm.

The step of selectively exposing the resist film may be carried out using light with various wavelengths such as ultraviolet rays and X-rays. For semiconductor resists, there may be generally used g-light, i-light, excimer laser (for example, XeCl, KrF, KrCl, ArF and ArCl) and soft X-rays. The exposure energy is, for example, about 0.1 to about 1,000 mJ/cm², preferably about 1 to about 100 mJ/cm².

### EXAMPLE

The present invention will be next described in more detail by way of examples and comparative examples. The scope of the present invention is, however, not limited to these examples in any way.

### Synthesis of Adamantane Derivatives

### Example 1

### (2-Hydroxy-2-adamantyl)methyl-2-methacrylate

Into a 1 L three-neck flask equipped with a thermometer, a condenser and a stirrer, 50 g (0.303 mole) of adamantane-2-spirooxirane and 500 ml of dimethylformamide were mixed. The mixture was stirred in the atmosphere of nitrogen until the solids were completely dissolved. After completion of dissolution, 11.5 g (0.0758 mole) of 1,8-diazabicycloundecene-7 and 51.2 mL of methacrylic acid were added and the mixture was stirred at 120° C. for 5 hours. After completion of the reaction, the reaction mixture was allowed to cool to room temperature (25° C.). Extraction was then carried out with ether. The extract was washed with water, concentrated and then added with hexane to crystallize the end product. Thus, 45 g of the end product was obtained in the form of a white solid (melting point: 87° C., yield: 59%).

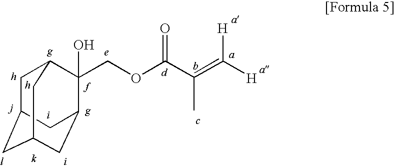

### Spectrum Data:

1. Nuclear magnetic resonance spectrum (solvent: CDCl₃, JNM-ECA500 manufactured by JEOL Ltd.)

¹H-NMR (500 MHz): 6.13 (1H, a″), 6.13 (1H, a′), 4.36 (2H, e), 2.08-2.26 (2H, g), 1.97 (3H, c), 1.71-1.78 (10H, h, i, j, k), 1.55-1.58 (2H, l).

¹³C-NMR (125 MHz): 18.42 (c), 27.12 (j or k), 27.29 (j or k), 27.12 (j or k), 32.79 (h or j), 34.38 (h or j), 35.21 (g), 38.00 (l), 69.39 (e), 74.22 (f), 125.85 (a), 136.17 (b), 167.50 (d).

2. Gas chromatograph-mass analysis spectrum (GC-MS-QP2010 manufactured by Shimadzu Corporation)

GC-MS: 151 (100%), 133 (6.3%), 91 (16.0%), 79 (9.6%), 69 (13.8%).

### Example 2

### [2-(methacryloxy)-2-adamantyl]methyl methacrylate

Into a 1 L three-neck flask equipped with a thermometer, a condenser and a stirrer, 50 g (0.303 mole) of adamantane-2-spirooxirane and 500 ml of dimethylformamide were mixed. The mixture was stirred in the atmosphere of nitrogen until the solids were completely dissolved. After completion of dissolution, 11.5 g (0.0758 mole) of 1,8-diazabicycloundecene-7 and 134.5 ml of methacrylic anhydride were added and the mixture was stirred at 120° C. for 12 hours. After completion of the reaction, the reaction mixture was allowed to cool to room temperature (25° C.). Extraction was then carried out with ether. The extract was washed with water and concentrated to obtain 50 g of the end product in the form of a white solid (melting point: 41.0° C., yield: 52%).

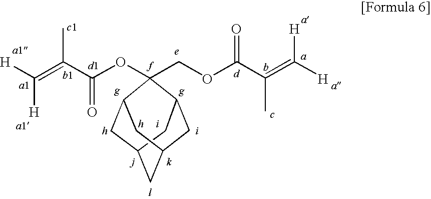

### Spectrum Data:

1. Nuclear magnetic resonance spectrum (solvent: CDCl₃, JNM-ECA500 manufactured by JEOL Ltd.)

¹H-NMR (500 MHz): 6.08 (2H, a″ and a1″), 5.53 (2H, a′ and a1′), 4.88 (2H, e), 2.52 (2H, g), 1.94 (6H, c and c1), 1.71-1.86 (10H, h, i, j, k), 1.55-1.58 (2H, l).

¹³C-NMR (125 MHz): 18.25, 18.42 (c1 or c), 26.90 (j or k), 32.22 (j or k), 32.50 (j or k), 34.11 (h or j), 34.44 (h or j), 34.73 (g), 38.01 (l), 63.03 (e), 86.11 (f), 124.73, 125.58 (a1 or a), 136.22, 137.54 (b1 or b), 166.13, 167.00 (d1 or d).

2. Gas chromatograph-mass analysis spectrum (GC-MS-QP2010 manufactured by Shimadzu Corporation)

GC-MS: 232 (19.3%), 219 (6.5%), 204 (14.2%), 163 (3.6%), 135 (2.9%), 69 (100%).

### Preparation of Cured Products and Evaluation of their Properties

### Example 3

A curable composition was prepared by mixing 60 parts of the adamantane derivative obtained in Example 1, 40 parts of methyl methacrylate and 1 part of benzoin butyl ether as a polymerization initiator. The composition was irradiated with UV rays at a light amount of 2,000 mJ/cm² and cured. The evaluation results of properties (1) to (5) of the thus obtained cured product are shown in Table 1.

### Example 4

A curable composition was prepared by mixing 100 parts of the adamantane derivative obtained in Example 2 and 1 part of benzoin butyl ether as a polymerization initiator. The composition was irradiated with UV rays at a light amount of 2,000 mJ/cm² and cured. The evaluation results of the physical properties (1) to (5) of the thus obtained cured product are shown in Table 1.

### Comparative Example 1

A composition was prepared by mixing 100 parts of methyl methacrylate and 1 part of benzoin isobutyl ether as a polymerization initiator. The composition was irradiated with UV rays and cured. The evaluation results of the physical properties (1) to (5) of the thus obtained cured product are shown in Table 1.

The evaluation of physical properties was carried out as follows.

(1) Glass transition temperature (° C.): Tg

A cured specimen (5 mg) was placed in an aluminum vessel and heated using a differential scanning calorimeter (DSC-7, manufactured by PerkinElmer, Inc.) at a rate of 10° C./min from 0° C. The glass transition temperature was determined from a discontinuous point observed in the obtained thermal flux curve.

(2) Thermal decomposition temperature (° C.): Td (5%)

A cured specimen (5 mg) was placed in an aluminum vessel and heated using a simultaneous thermogravimetric differential thermal analyzer (TG/DAT6000 manufactured by SII Nano Technology Inc.) at a rate of 5° C./min from 25° C. to 600° C. to obtain a mass change curve. The thermal decomposition temperature was determined from the temperature in the mass change curve at which the mass was reduced by 5%. Heat resistance is excellent when the glass transition temperature and thermal decomposition temperature are high.

(3) Total light transmittance (%)

A specimen having a thickness of 3 mm was measured in accordance with HS K7105 at the measurement wave length of 400 nm. A spectrophotometer UV-3100S, manufactured by Shimadzu Corporation, was used as the measuring instrument.

(4) Water absorption (%)

From a difference between the mass of a test piece (size: 30×30×3 mm) dried at 100° C. for 24 hours and the mass of the test piece immersed in water at 80° C. for 3 hours after the drying, the water absorption was calculated.

(5) Curing shrinkage (%)

The curing shrinkage was calculated from the specific gravities of a curable composition before and after curing.

TABLE 1

| | | | Comparative |
|---|---|---|---|
| | Example 3 | Example 4 | Example 1 |
| Glass transition temperature (° C.) | 107< | 163< | 108 |
| Thermal decomposition | 199 | 232 | 130 |
| temperature (° C.) | | | |
| Total light transmittance (%) | 93 | 93 | 90 |
| Water absorption (%) | 0.25 | 0.1 | 0.3 |
| Curing shrinkage (%) | 7 | 6.6 | 11 |

### Synthesis of (meth)acrylic-based polymer

### Example 5

In a flask, 12.5 parts of the monomer A, 23.4 parts of the monomer B, 17.0 parts of the monomer C, 4.1 parts of dimethyl 2,2′-azobis(isobutyrate) and 600 parts of methyl isobutyl ketone were charged in the atmosphere of nitrogen to form a monomer solution. In another flask, 200 parts of methyl isobutyl ketone were placed in the atmosphere of nitrogen and heated to 116° C. with stirring, to which was added dropwise the above-obtained monomer solution over 4 hours. After completion of the dropwise addition, the mixture was heated at 116° C. for 2 hours under reflux for aging and, thereafter, cooled to room temperature. Subsequently, the reaction liquid was added with 4,500 parts of a methanol/water mixed solvent (mixing ratio: 5:1) to precipitate the product. Such a precipitation procedure was repeated thrice in total to purify the product, thereby obtaining a copolymer P1 having a composition of monomer A: monomer B: monomer C of 18:38:44 (molar ratio), a weight average molecular weight (Mw) of 7,231 and a degree of dispersion (Mw/Mn) of 1.40.

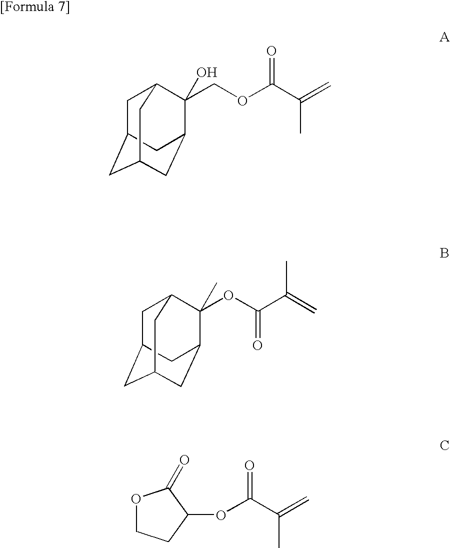

### Preparation of Posi-Type Resist Composition

### Example 6

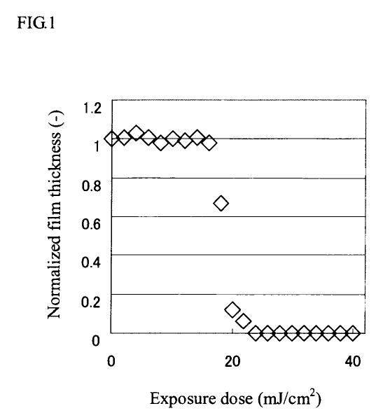

To 100 parts of the copolymer P1 obtained in Example 5, 5 parts of triphenylsulfonium nonafluorobutanesulfonate as a photoacid generator were added to obtain a resin composition. Then, in 90 parts of propylene glycol monomethyl ether acetate were dissolved 10 parts of the above-obtained resin composition to obtain a resist composition R1. The resist composition R1 was applied onto a silicon wafer and baked at 110° C. for 60 seconds to obtain a resist film. The thus obtained wafer was subjected to open exposure by a light of a wavelength of 248 nm with various different amounts of light exposure. Immediately after the exposure, the wafer was heated at 110° C. for 60 seconds and thereafter developed for 60 seconds using an aqueous tetramethylammonium hydroxide solution (2.38 wt %). The change of the film thickness relative to the amount of exposure is shown in Table 2 and in FIG. 1.

TABLE 2

| Exposure | Film thickness | Normalized |
|---|---|---|
| dose (mJ/cm²) | (nm) | film thickness (—) |
| 0 | 210 | 1.00 |
| 2 | 215 | 1.01 |
| 4 | 219 | 1.03 |
| 6 | 215 | 1.01 |
| 8 | 209 | 0.98 |
| 10 | 213 | 1.00 |
| 12 | 211 | 0.99 |
| 14 | 215 | 1.01 |
| 16 | 209 | 0.98 |
| 18 | 143 | 0.67 |
| 20 | 26 | 0.12 |
| 22 | 13 | 0.06 |
| 24 | 0 | 0 |
| 26 | 0 | 0 |
| 28 | 0 | 0 |
| 30 | 0 | 0 |
| 32 | 0 | 0 |
| 34 | 0 | 0 |
| 36 | 0 | 0 |
| 38 | 0 | 0 |
| 40 | 0 | 0 |

In Example 6 according to the present invention, a change in film thickness by amount of exposure is confirmed. Thus, it is confirmed that the resist composition R1 can serve to function as a photosensitive resin.

### INDUSTRIAL APPLICABILITY

The curable composition containing an adamantane derivative of the present invention having a group selected from an acrylate group, a methacrylate group and a trifluoromethacrylate group is capable of affording a cured product which is excellent in optical characteristics such as transparency and light resistance, durability such as long-term heat resistance, and electrical characteristics such as dielectric constant, is usable as a color resist material, as a sealant for opto-semiconductors, as an optical electronic member (such as an optical waveguide, a lens for optical communication, an optical film, etc.) and as an adhesive agent for them, and is also useful as a material for forming semiconductors such as a photoresist material for semiconductors and a anti-reflection coating for semiconductors.

1. An adamantane derivative represented by the following general formula (I): 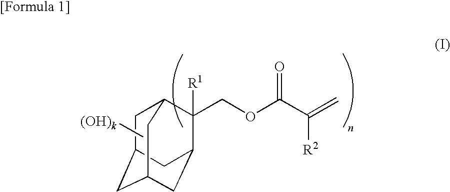
wherein R¹ represents a group selected from a hydroxyl group, an acrylate group, a methacrylate group and a trifluoromethacrylate group, R² represents a group selected from a hydrogen atom, a methyl group and a trifluoromethyl group, k is an integer of 0 to 4 and n is an integer of 1 to 6.

2. The adamantane derivative as recited in claim 1, wherein k in the general formula (I) is zero.

3. A process for producing the adamantane derivative according to claim 1, comprising reacting an epoxyadamantane with a compound selected from acrylic acid, methacrylic acid, trifluoromethacrylic acid, acrylic anhydride, methacrylic anhydride and trifluoromethacrylic anhydride.

4. A curable composition comprising the adamantane derivative according to claim 1, and a polymerization initiator.

5. A cured product obtained by curing the curable composition according to claim 4 by heating or by light irradiation.

6. A photoresist material using the adamantane derivative according to claim 1.

7. A color resist material using the adamantane derivative according to claim 1.

8. A (meth)acrylic-based polymer containing a monomer unit based on the adamantane derivative according to claim 1.

9. A resist composition comprising the (meth)acrylic polymer according to claim 8.

10. A resist pattern forming method, comprising the steps of forming a resist film on a substrate using the resist composition according to claim 9, selectively light-exposing the resist film, and subjecting the selectively light-exposed resist film to an alkali development treatment to form a resist pattern.
